(19) United States
(12) Patent Application Publication
(10) Pub. No.: US 2026/0240955 A1
(43) Pub. Date: Aug. 20, 2026

(54) PHARMACEUTICAL COMPOSITIONS OF LIRAGLUTIDE FOR INTRANASAL APPLICATION

(71) Applicant: Renaissance Lakewood, LLC, Lakewood, NJ (US)

(72) Inventor: Rohit Mulik, Lakewood, NJ (US)

(21) Appl. No.: 19/544,141

(22) Filed: Feb. 19, 2026

Related U.S. Application Data
(60) Provisional application No. 63/760,274, filed on Feb. 19, 2025.

(51) Int. Cl. A61K 38/26; A61K 9/00; A61K 47/10; A61K 47/26

(52) U.S. Cl. CPC A61K 38/26; A61K 9/0043; A61K 47/10; A61K 47/26

(57) ABSTRACT

The disclosure provides compositions for Liraglutide drug substance for intranasal application that meets desired physicochemical stability while maintaining solubility of the active pharmaceutical ingredient for a requisite duration. The present disclosure sets forth examples of a Liraglutide drug substance with various excipients (solvents, preservative, stabilizers, penetration enhancers, complexing agents) to achieve desired product attributes for solubility, stability, and pH.

This patent application claims priority to and benefit of U.S. Provisional Application No. 62/760,274, filed with the United States Patent and Trademark Office on Feb. 19, 2025, and is herein incorporated by reference in its entirety.

### FIELD OF INVENTION

This disclosure relates to intranasal composition. More specifically, the present disclosure relates to pharmaceutical compositions of Liraglutide for intranasal administration.

### BACKGROUND AND SUMMARY

The present disclosure provides pharmaceutical compositions of Liraglutide an active pharmaceutical ingredient (API) for intranasal application that exhibit desired physicochemical properties with respect to solubility aggregation) and chemical and structural stability. Liraglutide solubility is highly dependent on pH. It is freely soluble in aqueous basic solutions above pH 7.0 but has very low solubility in acidic to neutral aqueous solutions. The solubility of Liraglutide decreases as the pH drops below 7.0, reaching its lowest point at a pH of 4.0 to 5.0. Intranasal pH in healthy individuals is reported to range between 5.3 and 7.0. The present invention discloses a composition with a pH of 7.0 and higher that provides desired physicochemical stability while being suitable for intranasal application. The present disclosure sets forth examples of Liraglutide compositions with various excipients (solvents, preservative, stabilizers, penetration enhancers complexing agents) to achieve desired product attributes for solubility, stability, and pH for intranasal administration.

In various embodiments, the pharmaceutical Liraglutide compositions for intranasal application include Propylene Glycol (PG) based compositions. Contemplated pharmaceutical compositions for a Liraglutide drug substance for intranasal application may further include Glycerin based compositions. Contemplated compositions for a Liraglutide drug substance for intranasal application may further include Glycerin-Water based compositions. Contemplated compositions for a Liraglutide drug substance for intranasal application may further include Glycerin-trehalose based compositions. Contemplated Liraglutide compositions for intranasal application may further include compositions with 7.5% v/v Glycerin. Contemplated compositions for a Liraglutide drug substance for intranasal application may further include compositions with 11.5% v/v Glycerin. Contemplated compositions for a Liraglutide drug substance for intranasal application may further include compositions with 11.66% v/v Glycerin. Contemplated compositions for a Liraglutide drug substance for intranasal application may further include compositions with trehalose. Contemplated compositions for a Liraglutide drug substance for intranasal application may further include compositions with high molecular weight polyethylene glycol (e.g., PEG 3350). Contemplated pharmaceutical compositions for a Liraglutide drug substance for intranasal application include compositions having a pH of between 7.0 and 8.4, including compositions having a pH of 7.0 to 8.0, compositions having a pH of 7.2 to 8.0, compositions having a pH of 7.4 to 8.0, compositions having a pH of 7.4 to 7.6, compositions having a pH of 7.5 to 8.0, compositions having a pH of 7.5 to 7.6, compositions having a pH of 7.3 to 7.7, compositions having a pH of 7.0 to 7.8, compositions having a pH of 7.2 to 7.8, compositions having a pH of 7.5 to 7.9, compositions having a pH of 7.5 to 8.4, and compositions having a pH of 7.0 to 7.5. Contemplated pharmaceutical compositions for a Liraglutide drug substance for intranasal application include compositions having a pH of 7.5+/−0.4, compositions having a pH of 7.5+/−0.3, compositions having a pH of 7.5+/−0.2, compositions having a pH of 7.5+/−0.1, and compositions having a pH of 7.5.

A pharmaceutical composition for intranasal administration of the present disclosure may comprise an intranasal Liraglutide composition comprising water, one or more mixed solubilizing agent systems, one or more stabilizers, a pH of 7.0 to 8.4. In examples, the pharmaceutical composition for intranasal administration and/or the intranasal Liraglutide composition may comprise a pH of 7.0 to 8.4, a pH of 7.0 to 8.0, a pH of 7.2 to 8.0, a pH of 7.4, a pH of 7.5, a pH of 7.4 to 8.0, a pH of 7.5 to 8.0, a pH of 7.0 to 7.8, a pH of 7.3 to 7.7, a pH of 7.4 to 7.7, a pH of 7.4 to 7.6, a pH of 7.5 to 7.7, a pH of 7.5 to 7.6, a pH of 7.4 to 7.9, a pH of 7.5 to 7.9, a pH of 7.4 to 8.4, or a pH of 7.5 to 8.4. In examples, the pharmaceutical composition for intranasal administration and/or the intranasal Liraglutide composition may comprise a pH of 7.5+/−of 0.4, a pH of 7.5+/−0.3, a pH of 7.5+/−0.2, or a pH of 7.5+/−0.1. The pH may be maintained with or without a buffer such as, for example, a phosphate buffer. The one or more mixed solubilizing agent systems of the above examples may comprise solubilizing agents. More specifically, the one or more mixed solubilizing agent systems may comprise solubilizing agents such as PG, Glycerin, and high molecular weight polyethylene glycol (PEG). The high molecular weight polyethylene glycol may be polyethylene glycol 3350 (PEG 3350). For example, the mixed solubilizing agent system may comprise 7.5% v/v or 7.8% w/w PG, 7.5% v/v or 9.45% w/w Glycerin, 1.0% w/v PEG-3350 as solubilizing agents, and 2.5% w/v trehalose as the one or more stabilizers. Optionally, preservative and/or permeation enhancers may be further used in the Liraglutide composition of the pharmaceutical composition. A preservative used in the pharmaceutical composition may be Benzalkonium chloride (BZK). The preservative concentration may be up to 0.08% w/v of BZK. In the above examples, the one or more mixed solubilizing agent systems comprises between 5% v/v or 5.2% w/v and 15% v/v or 15.6% w/v of Propylene Glycol (PG), between 5% v/v or 6.3% w/v and 15% v/v or 18.9% w/v of Glycerin, between 0.3% w/v and 1.0% w/v PEG 3350 and between 0.25% w/v and 10% w/v of trehalose. As noted, the Liraglutide composition may further comprise, optionally, a preservative concentration of up to 0.08% w/v and the preservative may be BZK.

In the above examples, the intranasal Liraglutide composition may be maintained at 2-8° C. up to at least 9 months with a total Liraglutide impurities of less than 10%. In the above examples, the intranasal Liraglutide composition exhibits less than 10% total Liraglutide impurities when stored at 2-8° C. for up to at least 9 months. In the above examples, the intranasal Liraglutide composition exhibits less than 5% total Liraglutide impurities when stored at 2-8° C. for up to at least 9 months. In the above examples, the intranasal Liraglutide composition exhibits less than 10% total Liraglutide impurities when stored at 2-8° C. for up to at least 15 months. In the above examples, the intranasal Liraglutide composition exhibits less than 5% total Liraglutide impurities when stored at 2-8° C. for up to at least 15 months. In the above examples, the intranasal Liraglutide composition maintains an assay between 90.0-110.0% when stored at 2-8° C. for up to at least 9 months.

Also disclosed is an intranasal Liraglutide composition comprising: water, between 5% and 15% v/v or 5.2% w/v and 15.6% w/v PG, between 5% and 15% v/v or 6.3% w/v and 18.9% w/v Glycerin, between 0.3% w/v and 1.0% w/v PEG 3350, between 0.25% w/v and 10% w/v trehalose, a pH of 7.2 to 7.8 maintained with a phosphate buffer, and optionally up to 0.08% w/v of BZK.

The foregoing and other objects, features, and advantages of the examples will be apparent from the following more detailed descriptions of particular examples.

### BRIEF DESCRIPTION OF THE DRAWINGS

Reference is made to the accompanying drawings in which particular examples and further benefits of the examples are illustrated as described in more detail in the description below, in which:

FIG. 1 is a Shelf Life Plot of Representative Composition Lot 1, in accordance with an example of this disclosure.

FIG. 2 is a Shelf Life Plot of Representative Composition Lot 2, in accordance with an example of the disclosure.

### DETAILED DESCRIPTION

The present disclosure provides pharmaceutical compositions for Liraglutide drug substance for intranasal application that meet desired physicochemical stability while maintaining solubility of the Liraglutide for a requisite duration. The present disclosure also provides pharmaceutical compositions for Liraglutide drug substances for intranasal application that provides a physiological pH, suitable for intranasal application. The present disclosure sets forth examples of a Liraglutide drug substance with various excipients (solvents, preservative, stabilizers, penetration enhancers, complexing agents) to achieve desired product attributes for solubility, stability, and pH.

The solubility and stability of the Liraglutide drug substance pharmaceutical compositions for intranasal application at various pH levels were studied under various storage conditions.

Embodiments of a Liraglutide drug substance compositions of the present disclosure may include the addition of one or more excipients including, but not limited to, benzyl alcohol (BA), Ethanol, PEG 400, methoxy Polyethylene Glycol 350 (mPEG-350), PG, Phenylethyl Alcohol (PEA), and Vitamin E (Vit E). For example, solvent miscibility was assessed by testing solvents like BA, Ethanol, PEG 400, mPEG-350, PG, PEA with water to determine their compatibility and their ability to form homogenous solutions. As used here and unless indicated otherwise, water is Milli-Q® ultrapure water. The compositions may be binary mixtures, tertiary mixtures, quaternary mixtures, or the like, specific examples of which are described below.

The examples of Liraglutide drug substance compositions herein were additionally tested at a variety of concentrations of the Liraglutide drug substance using one or more excipients. The results were recorded by the specific examples, which are further described below. The Liraglutide drug substance compositions herein may or may not additionally include one or more preservatives. Preservatives, as used herein, include but are not limited one or more of BZK, BA, PEA, Chlorobutanol, and Potassium Sorbate. One or more of Hydroxypropyl-β-Cyclodextrin (HP-β-CD), Cyclopentadecanolide (CPD) and Dodecyl Maltoside (DDM) may, additionally, or alternatively, be relied on as permeation enhancer. One or more stabilizers may comprise trehalose, Dextran, trehalose, Sorbitol, Sodium Glycocholate, Poloxamer 188, and/or Cyclodextrins. Preferably, the stabilizer may comprise trehalose.

Exemplary Liraglutide drug substance compositions include the following excipients with PG concentrations ranging from 5% to 15% (v/v) or 5.2 to 15.6% w/v, glycerin concentrations ranging from 5% to 15% (v/v) or 6.3 to 18.9% w/v, PEG 3350 concentrations ranging from 0.3% to 1.0% (w/v), trehalose concentrations ranging from 0.25% to 10% (w/v), and, optionally, Benzalkonium chloride concentration of up to 0.08% (w/v).

The order of additions to the Liraglutide drug substance compositions also may be varied. In some examples, the order of addition included: forming the solution first, adding the drug substance thereafter, and adding preservatives and/or permeation enhancer after the drug substance, as required. In some examples, the order of addition included: forming the solution first, adding preservatives and/or permeation enhancers after forming the solution, as required, and adding the drug substance after adding preservatives and/or permeation enhancers. The order of addition, and their impact, were further adjusted as noted by the specific examples as further described below. Mixing durations are further developed by the specific examples as further described below.

For the examples herein, VWR® Multi-Position Hotplate Stirrers were employed with an appropriately sized magnetic stir bead. pH was measured using a Mettler Toledo Seven Excellence S400 pH meter with an InLab® Micro Pro-ISM pH Sensor.

Compositions at around a pH 7.5 were studied based on the previous observations of better physical stability of the compositions at around the pH of 7.5 as compared to pH of below 7.0 and/or a pH of 6.5.

Similar compositions were prepared using pH 7.5 phosphate buffers. 50% of the solution was filtered through a 0.22 μm hydrophilic Polyvinylidene Fluoride (PVDF) filter and both the unfiltered and filtered solutions were stored at room temperature (RT) and 2-8° C.

A composition (unfiltered and filtered solution) with 11.5% v/v Glycerin was found to be physically stable when visually observed for any precipitation or aggregation at 2-8° C. for 7 months and at RT, the unfiltered and unfiltered solution was found to be stable for ~2 months and ~7.5 months respectively (see Table 1).

Other combinations of Glycerin with Tween-20 in the presence of BZK-DDM were found to be physically stable when visually observed for any precipitation or fibrillation for ~2.5 months (unfiltered) and ~3 months (filtered) at 2-8° C. and at RT, the unfiltered solution was found to have less stability (52 days) compared to the filtered solution, which was found to be physically stable for ~2 months (Table 1). Glycerin with 0.25% w/v trehalose were clear at both RT and 2-8° C. with slight fibers observed for unfiltered solution and no fibers observed for the filtered solution (Table 1). Glycerin with Tween-20 and BZK was found to be physically stable when visually observed for any precipitation or aggregation at 2-8° C. for ~5 months and at RT for ~2 months. (see Table 1).

Other compositions with 11.5% v/v or 14.5% w/v Glycerin were studied in the presence of DDM, the unfiltered solution was found to be physically stable when visually observed for any precipitation or aggregation for ~1 month and the filtered solution was physically stable for ~6.5 months, at 2-8° C. At RT, filtration helped improve the solution's stability where it was clear for ~2.5 months (see Table 1) while the unfiltered solution was found to be physically stable for only 21 days. The addition of 0.25% w/v of trehalose to a similar composition (Glycerin-DDM) resulted in improved stability where the unfiltered and filtered solutions at 2-8° C. were found to be physically stable when visually observed for any precipitation or aggregation for ~3 months and ~5 months respectively and for ~1.5 months and ~3.5 months at RT respectively (see Table 1) indicating the stabilizing effect of trehalose. Unfiltered and filtered solutions with 11.5% v/v or 14.5% w/v Glycerin with BZK-trehalose were found to be physically stable when visually observed for any precipitation or aggregation for ~4.5 months at both 2-8° C. and at RT, (see Table 1). The addition of both DDM and BZK did not appear to improve the stability when visually observed for any precipitation or aggregation at RT, where the unfiltered solution was stable for only ~2.5 months and filtered one for ~4 months. At 2-8° C., similar stability was observed where both the unfiltered and filtered solutions were found to be stable for ~4.5 months. (see Table 1).

Experiments were performed with 11.5% v/v or 12.0% w/v PG with 10 mg/mL, 15 mg/mL, and 20 mg/mL API concentration. Both unfiltered and filtered solutions for all three compositions were clear for ~1-1.5 months at RT, with the filtered solution exhibiting longer physical stability than the unfiltered solutions. Also, unfiltered and filtered solutions of 10 mg/mL & 15 mg/mL API concentrations with 11.5% v/v or 12.0% w/v PG were found to be stable when visually observed for any precipitation or aggregation for ~4.5 months at 2-8° C. Unfiltered and filtered solutions at 20 mg/mL composition were found to be physically stable when visually observed for any precipitation or aggregation for 3 months and ~3.5 months at 2-8° C. respectively.

Other sets of studies were performed using lower and higher concentrations of PG with water at 20 mg/mL API concentration. 7.5% v/v or 7.8% w/v and 15% v/v or 15.6% w/v PG was used with water. All the solutions were clear at 2-8° C. At RT, the filtered solutions were clear for ~2 months (see Table 8) and the unfiltered solutions were clear for ~1 month.

Unfiltered PG (8.7% v/v or 9.0% w/v)—mPEG 350 (2.9% w/v) solution of 20 mg/mL API precipitated in 30 days at RT and in 6 days at 2-8° C. while the filtered solution was physically stable at RT for ~3.5 months (see Table 1) and was clear at 2-8° C. for ~5 months. While similar compositions at 10 mg/mL and 15 mg/mL were found to be physically stable when visually observed for any precipitation or aggregation for ~6 months at 2-8° C. At RT, 15 mg/mL solutions exhibited stability for 3.5 months, while 10 mg/mL solutions were stable for 4 months (see Table 1).

The unfiltered solution with PG-mPEG 350 and 0.25% w/v trehalose was physically stable when visually observed for any precipitation or aggregation for ~3 months at 2-8° C. and for 2 months at RT (see Table 1), while the filtered solution was stable for ~5 months at 2-8° C. and ~3.5 months at RT (see Table 1). Adding trehalose to the PG-mPEG 350 solubilizing agent system improved the stability of the unfiltered solution at both storage temperatures of 2-8° C. and RT.

Solutions (filtered and unfiltered) with various concentrations of trehalose (2.5%, 5%, and 10% w/v) in combination with 11.5% v/v or 12.0% w/v PG were clear for ~6.5 months at 2-8° C., except the filtered solution with 5% trehalose which precipitated in ~4 months. At RT, the unfiltered solution with the least concentration of trehalose (2.5%) exhibited the shortest stability of ~2 months for both unfiltered and filtered solutions (Table 1). Whereas the filtered and unfiltered solutions at 5% trehalose were physically stable for ~3 months for filtered solution and ~2.5 months for the unfiltered solution (see Table 1). Maximum stability was observed for solutions with 10% trehalose, the filtered solution was clear for ~4.5 months and the unfiltered solution was clear for ~2.5 months (see Table 1). Composition with 2.5% w/v trehalose with 7.5% v/v PG was found to be stable with a clear solution observed for ~4.5 months both the unfiltered and filtered solutions at 2-8° C. At RT, the unfiltered solution exhibited haziness after ~1.5 months and the filtered solution was clear for ~3 months (see Table 1).

Unfiltered solution with 1% w/v PEG 3350 was physically stable for a lesser time at RT & 2-8° C. At RT, the unfiltered solution precipitated in 7 days and precipitated in 24 days at 2-8° C. Crystals were observed in the filtered solution after ~1.5 months at RT, while the filtered solution was stable for ~3 months at 2-8° C.

Filtered solutions of 25 mg/mL and 200 mg/mL of HP-β-CD in combination with both BZK (up to 0.08% w/v) and DDM (up to 0.2% w/v) were clear at 2-8° C. for ~3.5 months and ~4.5 months respectively. At RT, these solutions gelled after ~1 month. Haziness was observed in the unfiltered solution with 25 mg/mL HP-β-CD after 8 days at RT, while it was physically stable for ~1 month at 2-8° C. Unfiltered solution with 200 mg/mL HP-β-CD showed slightly longer stability of ~2 months at 2-8° C. and ~1 month at RT.

Longer stability of ~4 months at 2-8° C. and for ~2 months at RT was observed for the filtered solution of 10% w/v trehalose with 0.1% w/v DDM (see Table 1). Unfiltered solutions precipitated in ~2 months and ~1 month at 2-8° C. and RT respectively.

The compositions at pH 7.5 which were found to be stable for over ~2 months at RT are reported in Table 1.

TABLE 1
Physical Stability Results (Visual Observation for Precipitation/Fibrillation)
for Compositions at pH 7.5

| | Observations | Observations |
|---|---|---|
| Composition | at 2-8° C. | at RT |
| Milli-Q ® Water + PG + mPEG-350 + | Slight ppts observed | Slight ppts observed |
| Phosphate Buffer, pH-7.5, Filtered, | after ~5 months | after ~3.5 months |
| 20 mg/mL API | | |
| Milli-Q ® Water + PG + mPEG-350 + | Slight ppts observed | Ppts observed |
| trehalose + Phosphate Buffer, pH 7.5, | after ~3 months | after ~2 months |
| Unfiltered, 20 mg/mL API | | |
| Milli-Q ® Water + PG + mPEG-350 + | Slight ppts observed | Slight ppts observed |
| trehalose + Phosphate Buffer, pH 7.5, | after ~5 months | after ~3.5 months |
| Filtered, 20 mg/mL API | | |
| Milli-Q ® Water + Glycerin (11.66%) + | Slight ppts observed | Ppts observed |
| Phosphate Buffer, pH-7.5, Unfiltered, | after ~9.5 months | after ~2 months |
| 20 mg/mL API | | |
| Milli-Q ® Water + Glycerin (11.66%) + | Slight ppts observed | Slight ppts observed |
| Phosphate Buffer, pH-7.5, Filtered, | after ~9.5 months | after ~7.5 months |
| 20 mg/mL API | | |
| Milli-Q ® Water + Glycerin (11.66%) + | Stable for 12 months | Ppts observed |
| trehalose (0.25%) + Phosphate Buffer, | | after ~6.5 months |
| pH-7.5, Unfiltered, 20 mg/mL API | | |
| Milli-Q ® Water + Glycerin (11.66%) + | Stable for 12 months | Gelling observed |
| trehalose (0.25%) + Phosphate Buffer, | | after 10 months |
| pH-7.5, Filtered, 20 mg/mL API | | |
| Milli-Q ® Water + Glycerin (7.5%) + | Stable for 9 months | Slight ppts observed |
| T20 + BZK (0.08%) + Phosphate Buffer, | | after 2 months |
| pH 7.5, Filtered, 20 mg/mL API | | |
| Milli-Q ® Water + Glycerin (11.66%) + | Stable for 12 months | Slight ppts observed |
| trehalose (0.25%) + DDM + Phosphate | | after ~3.5 months |
| Buffer, pH-7.5, Filtered, 20 mg/mL API | | |
| Milli-Q ® Water + Glycerin (11.66%) + | Ppts observed | Slight ppts observed |
| T-20 + BZK (0.08%) + DDM + Phosphate | after ~3 months | after ~2 months |
| Buffer, pH-7.5, Filtered, 20 mg/mL API | | |
| Milli-Q ® Water + Glycerin (11.66%) + | Stable for 7 months | Slightly hazy |
| DDM + Phosphate Buffer, pH-7.5, | | solution observed |
| Filtered, 20 mg/mL API | | after ~2.5 months |
| Milli-Q ® Water + Glycerin (11.66%) + | Stable for 11 months | Viscous solution |
| trehalose (0.25%) + BZK (0.08%) + | | observed after ~7.5 |
| Phosphate Buffer, pH-7.5, Unfiltered, | | |
| 20 mg/mL API | | |
| Milli-Q ® Water + Glycerin (11.66%) + | Stable for 11 months | Gelling observed |
| trehalose (0.25%) + BZK (0.08%) + | | after ~8 months |
| Phosphate Buffer, pH-7.5, Filtered, | | |
| 20 mg/mL API | | |
| Milli-Q ® Water + Glycerin (11.5%) + | Stable for 11 months | Slightly hazy |
| trehalose (0.25%) + DDM + | | solution observed |
| BZK (0.08%) + Phosphate Buffer, | | after ~2.5 months |
| pH-7.5, Unfiltered, 20 mg/mL API | | |
| Milli-Q ® Water + Glycerin (11.5%) + | Stable for 11 months | Slight ppts observed |
| trehalose (0.25%) + DDM + | | after 4 months |
| BZK (0.08%) + Phosphate Buffer, | | |
| pH-7.5, Filtered, 20 mg/mL API | | |
| Milli-Q ® Water + PG (15%) + | Slight ppts observed | Slight ppts observed |
| Phosphate Buffer, pH 7.5, Filtered, | after 10 months | after ~2 months |
| 20 mg/mL API | | |
| Milli-Q ® Water + PG (7.5%) + | Crystals observed | Slight ppts observed |
| Phosphate Buffer, pH 7.5, Filtered, | after 8 months | after ~2 months |
| 20 mg/mL API | | |
| Milli-Q ® Water + PG (11.66%) + | Slight ppts observed | Ppts observed |
| trehalose (2.5%) + Phosphate Buffer, | after 9.5 months | after ~2 months |
| pH 7.5, Filtered, 20 mg/mL API | | |
| Milli-Q ® Water + PG (11.66%) + | Slight ppts observed | Slight ppts observed |
| trehalose (5%) + Phosphate Buffer, | after 11 months | after ~2.5 months |
| pH 7.5, Unfiltered, 20 mg/mL API | | |
| Milli-Q ® Water + PG (11.66%) + | Slight ppts observed | Slight ppts observed |
| trehalose (5%) + Phosphate Buffer, | after ~4.5 months | after ~3 months |
| pH 7.5, Filtered, 20 mg/mL API | | |
| Milli-Q ® Water + PG (11.66%) + | Stable for 11 months | Slight ppts observed |
| trehalose (10%) + Phosphate Buffer, | | after ~2.5 months |
| pH 7.5, Unfiltered, 20 mg/mL API | | |
| Milli-Q ® Water + PG (11.66%) + | Slight ppts observed | Slight ppts observed |
| trehalose (10%) + Phosphate Buffer, | after 10 months | after ~4.5 months |
| pH 7.5, Filtered, 20 mg/mL API | | |
| Milli-Q ® Water + PG (7.5%) + | Slight ppts observed | Ppts observed |
| trehalose (2.5%) + Phosphate Buffer, | after ~4.5 months | after ~3 months |
| pH 7.5, Filtered, 20 mg/mL API | | |
| Milli-Q ® Water + PG (11.5%) + | Stable for 11 months | Slight ppts observed |
| mPEG-350 + Phosphate Buffer, pH 7.5,- | | after ~2.5 months |
| Unfiltered, 10 mg/mL API | | |
| Milli-Q ® Water + PG (11.5%) + | Stable for 11 months | Slight ppts observed |
| mPEG-350 - pH 7.5-10mg/mL - filtered | | after 4 months |
| Milli-Q ® Water + PG (11.5%) + | Stable for 11 months | Slight ppts observed |
| mPEG-350 + Phosphate Buffer, pH-7.5, | | after ~2 months |
| Unfiltered, 15 mg/mL API | | |
| Milli-Q ® Water + PG (11.5%) + | Stable for 11 months | Slight ppts |
| mPEG-350 + Phosphate Buffer, pH-7.5, | | after ~3.5 months |
| Filtered, 15mg/mL API | | |
| Milli-Q ® Water + trehalose (10%) + | Slight ppts observed | Slight ppts observed |
| DDM + Phosphate Buffer, pH 7.5, | after 4 months | after ~2 months |
| Filtered, 20 mg/mL API | | |

It was determined that the stability of the API is pH-dependent, indicating that compositions with pH 7.5 exhibited longer stability compared to the compositions at pH 6.5 and pH 7.0, of which filtered solutions were slightly more stable than unfiltered solutions.

Compositions with mixed solubilizing agents were further studied based on previous observations indicating the better stability of compositions with mixed solubilizing agents.

A mixed solubilizing agent system (PG-Glycerin) was used in addition to PEG 3350 & trehalose and water, with and without BZK-DDM. Concentrations were decided based on the stability studies observed with individual solvents/excipients. 7.5% v/v or 7.8% w/v of PG and 7.5% v/v or 9.45% w/v of Glycerin was used with 1% w/v PEG 3350 and 2.5% w/v trehalose at pH 7.0 and pH 7.5. All the solutions (pH 7.0 & pH 7.5) were clear for more than ~5 months at 2-8° C. (see Table 2).

At RT, the compositions at pH 7.0 exhibited shorter stability of ~2.5 months while at pH 7.5, the compositions were found to be stable for ~5 months Indicating the impact of pH on Liraglutide stability (see Table 2).

The addition of BZK and DDM to the above composition did not aid in increasing the stability of the compositions at RT. Compositions with pH 7.0 were stable for ~2 months and at pH 7.5 the unfiltered solution was physically stable for ~1.5 months and the filtered solution was clear for over ~2.5 months (Table 2). All the solutions at 2-8° C. were clear for over ~5 months except the filtered solution at pH 7.0, which precipitated in ~2.5 months indicating the impact of pH on Liraglutide stability.

TABLE 2
Physical Stability Results (Visual Observation for Precipitation/Fibrillation)
for Compositions with Mixed Solubilizing Agents.

| | Observations | Observations |
|---|---|---|
| Composition | at 2-8° C. | at RT |
| Milli-Q ® Water + 7.5% PG + 7.5% | Stable for 17.5 months | Slight ppts observed |
| Glycerin + 1% PEG 3350 + 2.5% | | after ~2.5 months |
| trehalose + Phosphate Buffer, pH 7.0, | | |
| Unfiltered, 20 mg/mL API | | |
| Milli-Q ® Water + 7.5% PG + 7.5% | Stable for 17.5 months | Slight ppts observed |
| Glycerin + 1% PEG 3350 + 2.5% | | after ~2.5 months |
| trehalose + Phosphate Buffer, pH 7.0, | | |
| Filtered, 20 mg/mL API | | |
| Milli-Q ® Water + 7.5% PG+ 7.5% | Stable for 17.5 months | Slight ppts observed |
| Glycerin + 1% PEG 3350 + 2.5% | | after 9 months |
| trehalose + Phosphate Buffer, pH 7.5, | | |
| Unfiltered, 20 mg/mL API | | |
| Milli-Q ® Water + 7.5% PG+ 7.5% | Stable for 17.5 months | Stable for 6.5 months |
| Glycerin + 1% PEG 3350 + 2.5% | | |
| trehalose + Phosphate Buffer, pH 7.5, | | |
| Filtered, 20 mg/mL API | | |
| Milli-Q ® Water + 7.5% PG+ 7.5% | Stable for ~10 months | Slight ppts observed |
| Glycerin + 1% PEG 3350 + 2.5% | | after 1.5 months |
| trehalose + 0.08% BZK + 0.2% | | |
| DDM + Phosphate Buffer, pH 7.5, | | |
| Unfiltered, 20 mg/mL API | | |
| Milli-Q ® Water + 7.5% PG+ 7.5% | Stable for ~10 months | Slight ppts observed |
| Glycerin + 1% PEG 3350 + 2.5% | | after ~2.5 months |
| trehalose + 0.08% BZK + 0.2% | | |
| DDM + Phosphate Buffer, pH 7.5, | | |
| Filtered, 20 mg/mL API | | |

Using these studies as a baseline, the chemical stability of Liraglutide compositions were evaluated. The Reverse Phase High-Performance Liquid Chromatography combined with Ultraviolet and Mass Spectrometry (RP-HPLC-UV-MS^{e}) approach was used with Waters™ UPLC CORTECS C18+ 1.6 μm column (3×150 mm), connected to BioPharmaSpec's Waters™ Acquity UPLC in-line with BioPharmaSpec's Waters™ Acquity PDA detector and Xevo G2-XS Q-TOF Mass Spectrometer for molecular weight analysis of Liraglutide samples in order to detect and/or identify peptide-related impurities (including UV detection) and quantify native Liraglutide content. More specifically, online Liquid Chromatography-Electrospray Ionization-Mass Spectrometry (LC/ES-MS) (Quadrupole Time-of-Flight (Q-TOF)) analysis was performed for assessment of Liraglutide related impurities (including UV detection).

The Following compositions were used for the preliminary analysis of the suitability of the developed method and also to evaluate the stability of developed compositions to identify potential stable compositions.

A Milli-Q® Water+Glycerin (11.66% v/v or 14.7% w/v)+trehalose (0.25% w/v)+BZK (0.08% w/v)+Phosphate Buffer, (pH-7.5, Unfiltered, 20 mg/mL) composition showed ~17% drop in Liraglutide assay and corresponding increase in peptide related impurities after 5 months storage at 25° C. indicating instability at accelerated storage condition. Total of 22 impurities were observed at ≥0.05 relative peak area (%) integrated by 215 nm UV detection.

A Milli-Q® Water+Glycerin (11.66% v/v or 14.7% w/v)+trehalose (0.25% w/v)+BZK (0.08% w/v)+Phosphate Buffer, (pH-7.5, Filtered, 20 mg/mL API) composition showed ~17% drop in Liraglutide assay and corresponding increase in peptide related impurities after 5 months storage at 25° C. indicating instability at accelerated storage condition. Total of 26 impurities were observed at ≥0.05 relative peak area (%) integrated by 215 nm UV detection. This sample was filtered through hydrophilic PVDF membrane syringe filter. Since the assay of this sample was similar to the unfiltered sample, it was concluded that there is no impact on Liraglutide assay upon filtration.

A Milli-Q® Water+PG (7.5% v/v or 7.8% w/v)+Glycerin (7.5% v/v or 9.45% w/v)+PEG 3350 (1% w/v)+trehalose (2.5% w/v)+Phosphate Buffer, (pH 7.5, Filtered, 20 mg/mL API) composition showed ~6% drop in Liraglutide assay and corresponding increase in peptide-related impurities after 3 months storage at 25° C. Total of 15 impurities were observed at ≥0.05 relative peak area (%) integrated by 215 nm UV detection which was less than the four other compositions tested. This result indicated that this composition has the potential to possess better stability at long-term storage of 2-8° C.

A Milli-Q® Water+Glycerin (11.5% v/v or 14.5% w/v)+Dextran (6% w/w)+trehalose (0.25% w/v)+BZK (0.08% w/v)+DDM (0.2% w/v)+Phosphate Buffer, (pH 7.5, Filtered, 20 mg/mL API) composition showed ~37% drop in Liraglutide assay and corresponding increase in peptide related impurities only after 2 months storage at 25° C. indicating significant instability at accelerated storage condition. Total of 33 impurities were observed at ≥0.05 relative peak area (%) integrated by 215 nm UV detection which was more than the four other compositions tested.

A Milli-Q® Water+Glycerin (11.66% v/v or 14.7% w/v)+trehalose (0.25% w/v)+BZK (0.08% w/v)+Phosphate Buffer, (pH-7.5, Filtered, 20 mg/mL API) composition showed ~10% drop in Liraglutide assay and corresponding increase in peptide related impurities after 5 months storage at 2-8° C. indicating significant instability at long-term storage condition. Total of 17 impurities were observed at ≥0.05 relative peak area (%) integrated by 215 nm UV detection.

The testing of these five compositions indicated that the presence of BZK and/or DDM results in increase in impurities and corresponding decrease in Liraglutide assay. Detailed impurities data for each of the above compositions follows in Table 3.1-Table 3.5, respectively, below.

TABLE 3.1
Milli-Q ® Water + Glycerin (11.66% v/v
or 14.7% w/v) + trehalose (0.25% w/v) + BZK
(0.08% w/v) + Phosphate Buffer, (pH-7.5, Unfiltered,
20 mg/mL) - After 5 Months at 25° C.

| Relative | Retention | Peak Area | |
|---|---|---|---|
| Retention | Time | (PA) | Peak Area |
| Time (RRT) | (RT) (min) | 215 nm | (PA) % |
| 0.64 | 38.2 | 13418 | 0.46 |
| 0.92 | 54.4 | 17078 | 0.59 |
| 1 | 59.3 | 2404716 | 82.89 |
| 1.13 | 67.2 | 127932 | 4.41 |
| 1.22 | 72.1 | 49918 | 1.72 |
| 1.24 | 73.8 | 13009 | 0.45 |
| 1.29 | 76.3 | 6550 | 0.23 |
| 1.32 | 78.1 | 22746 | 0.78 |
| 1.33 | 79.1 | 18315 | 0.63 |
| 1.37 | 81.4 | 24914 | 0.86 |
| 1.39 | 82.6 | 38639 | 1.33 |
| 1.43 | 84.5 | 18799 | 0.65 |
| 1.46 | 86.2 | 2041 | 0.07 |
| 1.46 | 86.8 | 2521 | 0.09 |
| 1.47 | 87.2 | 1782 | 0.06 |
| 1.48 | 87.6 | 2838 | 0.1 |
| 1.49 | 88.2 | 3964 | 0.14 |
| 1.49 | 88.5 | 22167 | 0.76 |
| 1.5 | 89.1 | 12183 | 0.42 |
| 1.51 | 89.5 | 22199 | 0.77 |
| 1.53 | 90.6 | 22273 | 0.77 |
| 1.54 | 91.5 | 2953 | 0.1 |
| 1.58 | 93.6 | 50292 | 1.73 |
| N = 22 | Sum | 2901250 | 100 |

TABLE 3.2
Milli-Q ® Water + Glycerin (11.66% v/v
or 14.7% w/v) + trehalose (0.25% w/v) + BZK
(0.08% w/v) + Phosphate Buffer, (pH-7.5, Filtered,
20 mg/mL API) - After 5 Months at 25° C.

| Relative | Retention | Peak Area | |
|---|---|---|---|
| Retention | Time | (PA) | Peak Area |
| Time (RRT) | (RT) (min) | 215 nm | (PA) % |
| 0.64 | 37.5 | 10265 | 0.34 |
| 0.75 | 43.6 | 1823 | 0.06 |
| 0.92 | 53.7 | 15235 | 0.51 |
| 1 | 58.4 | 2505000 | 83.06 |
| 1.13 | 65.9 | 13047 | 0.43 |
| 1.14 | 66.6 | 117409 | 3.89 |
| 1.21 | 70.6 | 7635 | 0.25 |
| 1.22 | 71.3 | 57602 | 1.91 |
| 1.25 | 73.1 | 11454 | 0.38 |
| 1.29 | 75.5 | 5965 | 0.2 |
| 1.33 | 77.4 | 23349 | 0.77 |
| 1.34 | 78.4 | 20440 | 0.68 |
| 1.38 | 80.8 | 2687 | 0.09 |
| 1.4 | 81.6 | 2305 | 0.08 |
| 1.41 | 82.3 | 43938 | 1.46 |
| 1.43 | 83.4 | 1958 | 0.06 |
| 1.45 | 84.4 | 24846 | 0.82 |
| 1.48 | 86.1 | 1821 | 0.06 |
| 1.49 | 86.7 | 2651 | 0.09 |
| 1.5 | 87.5 | 2612 | 0.09 |
| 1.51 | 88.2 | 3385 | 0.11 |
| 1.52 | 88.5 | 22849 | 0.76 |
| 1.52 | 89 | 13482 | 0.45 |
| 1.53 | 89.4 | 24505 | 0.81 |
| 1.55 | 90.5 | 24904 | 0.83 |
| 1.57 | 91.5 | 2932 | 0.1 |
| 1.6 | 93.5 | 51743 | 1.72 |
| N = 26 | Sum | 3015844 | 100 |

TABLE 3.3
Milli-Q ® Water + PG (7.5% v/v or 7.8% w/v) +
Glycerin (7.5% v/v or 9.45% w/v) + PEG 3350 (1% w/v) +
trehalose (2.5% w/v) + Phosphate Buffer, (pH 7.5, Filtered,
20 mg/mL API) - After 5 Months at 25° C.

| Relative | Retention | Peak Area | |
|---|---|---|---|
| Retention | Time | (PA) | Peak Area |
| Time (RRT) | (RT) (min) | 215 nm | (PA) % |
| 0.64 | 37.5 | 10535 | 0.38 |
| 0.92 | 53.7 | 8547 | 0.31 |
| 1 | 58.2 | 2599488 | 93.63 |
| 1.13 | 66 | 10650 | 0.38 |
| 1.23 | 71.6 | 26082 | 0.94 |
| 1.25 | 73.1 | 18693 | 0.67 |
| 1.35 | 78.6 | 7008 | 0.25 |
| 1.4 | 81.4 | 6146 | 0.22 |
| 1.41 | 82.4 | 32733 | 1.18 |
| 1.45 | 84.4 | 16770 | 0.6 |
| 1.49 | 86.7 | 1764 | 0.06 |
| 1.51 | 88.2 | 1764 | 0.06 |
| 1.52 | 88.5 | 11321 | 0.41 |
| 1.53 | 89.1 | 7099 | 0.26 |
| 1.54 | 89.5 | 7706 | 0.28 |
| 1.56 | 90.6 | 10044 | 0.36 |
| N = 15 | Sum | 2776349 | 100 |

TABLE 3.4
Milli-Q ® Water + Glycerin (11.5% v/v
or 14.5% w/v) + Dextran (6% w/w) + trehalose
(0.25% w/v) + BZK (0.08% w/v) + DDM (0.2%
w/v) + Phosphate Buffer,(pH 7.5, Filtered, 20
mg/mL API) - After 2 Months at 25° C.

| Relative | Retention | Peak Area | |
|---|---|---|---|
| Retention | Time | (PA) | Peak Area |
| Time (RRT) | (RT) (min) | 215 nm | (PA) % |
| 0.63 | 37.5 | 9115 | 0.41 |
| 0.74 | 44 | 8745 | 0.4 |
| 0.76 | 45.3 | 3683 | 0.17 |
| 0.9 | 53.9 | 12853 | 0.58 |
| 0.96 | 57.5 | 7555 | 0.34 |
| 0.98 | 58.4 | 23980 | 1.09 |
| 1 | 59.6 | 1380147 | 62.77 |
| 1.11 | 65.8 | 14655 | 0.67 |
| 1.14 | 67.9 | 9396 | 0.43 |
| 1.17 | 69.5 | 4079 | 0.19 |
| 1.18 | 70.4 | 255050 | 11.6 |
| 1.23 | 73 | 8070 | 0.37 |
| 1.26 | 75.2 | 7675 | 0.35 |
| 1.29 | 76.8 | 142607 | 6.49 |
| 1.31 | 78.3 | 25772 | 1.17 |
| 1.34 | 80.1 | 4904 | 0.22 |
| 1.36 | 80.7 | 4799 | 0.22 |
| 1.37 | 81.4 | 1156 | 0.05 |
| 1.38 | 82.4 | 31685 | 1.44 |
| 1.4 | 83.4 | 6590 | 0.3 |
| 1.42 | 84.4 | 17340 | 0.79 |
| 1.45 | 86.1 | 1976 | 0.09 |
| 1.45 | 86.4 | 2638 | 0.12 |
| 1.46 | 86.7 | 5938 | 0.27 |
| 1.46 | 87.2 | 1335 | 0.06 |
| 1.47 | 87.6 | 5231 | 0.24 |
| 1.48 | 87.9 | 4200 | 0.19 |
| 1.48 | 88.2 | 6061 | 0.28 |
| 1.49 | 88.5 | 14121 | 0.64 |
| 1.5 | 89.3 | 76097 | 3.46 |
| 1.52 | 90.5 | 74940 | 3.41 |
| 1.54 | 91.5 | 16634 | 0.76 |
| 1.55 | 92.3 | 4194 | 0.19 |
| 1.57 | 93.7 | 5374 | 0.24 |
| N = 33 | Sum | 2198594 | 100 |

TABLE 3.5
Milli-Q ® Water + Glycerin (11.66% v/v
or 14.7% w/v) + trehalose (0.25% w/v) + BZK
(0.08% w/v) + Phosphate Buffer, (pH-7.5, Filtered,
20 mg/mL API) - After 5 Months at 2-8° C.

| Relative | Retention | Peak Area | |
|---|---|---|---|
| Retention | Time | (PA) | Peak Area |
| Time (RRT) | (RT) (min) | 215 nm | (PA) % |
| 0.65 | 37.5 | 6022 | 0.17 |
| 0.93 | 53.6 | 12036 | 0.35 |
| 1 | 57.8 | 3120441 | 89.62 |
| 1.15 | 66.5 | 116933 | 3.36 |
| 1.24 | 71.7 | 29962 | 0.86 |
| 1.31 | 75.6 | 3510 | 0.1 |
| 1.35 | 77.7 | 11015 | 0.32 |
| 1.36 | 78.6 | 16489 | 0.47 |
| 1.4 | 81.1 | 10893 | 0.31 |
| 1.43 | 82.7 | 32302 | 0.93 |
| 1.46 | 84.5 | 5474 | 0.16 |
| 1.53 | 88.2 | 1762 | 0.05 |
| 1.53 | 88.5 | 21080 | 0.61 |
| 1.54 | 89 | 11044 | 0.32 |
| 1.55 | 89.5 | 13506 | 0.39 |
| 1.57 | 90.6 | 16536 | 0.47 |
| 1.58 | 91.5 | 1668 | 0.05 |
| 1.62 | 93.6 | 51282 | 1.47 |
| N = 17 | Sum | 3481956 | 100 |

Further, additional compositions were tested for chemical stability using Reverse Phase High-Performance Liquid Chromatography combined with Ultraviolet and Mass Spectrometry (RP-HPLC-UV-MSe) with Waters™ UPLC CORTECS C18+ 1.6 μm column (3×150 mm), connected to BioPharmaSpec's Waters™ Acquity UPLC in-line with BioPharmaSpec's Waters™ Acquity PDA detector and Xevo G2-XS Q-TOF Mass Spectrometer for molecular weight analysis of Liraglutide samples in order to detect and/or identify peptide-related impurities (including UV detection) and quantify native Liraglutide content for shortlisting the compositions for stability study. The following compositions were tested.

A Milli-Q® Water+Glycerin (11.66% v/v or 14.7% w/v)+trehalose (0.25% w/v)+BZK (0.08% w/v)+Phosphate Buffer, (pH 7.5, Filtered, 20 mg/mL API) composition showed 98.10% Liraglutide assay after 1 month storage at 2-8° C. (long-term stability) indicating stable composition. Only a total of 9 impurities were observed at ≥0.05 relative peak area (%) integrated by 215 nm UV detection.

A Milli-Q® Water+PG (7.5% v/v or 7.8% w/v)+Glycerin (7.5% v/v or 9.45% w/v)+PEG 3350 (1.0% w/v)+trehalose (2.5% w/v)+Phosphate Buffer, (pH 7.5, Filtered, 20 mg/mL API) composition showed 99.28% Liraglutide assay after 1 month storage at 2-8° C. (long-term stability) indicating stable composition. Only a total of 5 impurities were observed at ≥0.05 relative peak area (%) integrated by 215 nm UV detection.

A Milli-Q® Water+PG (7.5% v/v or 7.8% w/v)+Glycerin (7.5% v/v or 9.45% w/v)+PEG 3350 (1.0% w/v)+trehalose (2.5% w/v)+Phosphate Buffer, (pH 7.5, Filtered, 20 mg/mL API) composition showed 96.16% Liraglutide assay after 7 months storage at 2-8° C. (long-term stability) indicating stable composition. Total of 15 impurities were observed at ≥0.05 relative peak area (%) integrated by 215 nm UV detection.

A Milli-Q® Water+Glycerin (11.5% v/v or 14.5% w/v)+Dextran (6% w/v)+trehalose (2.5% w/v)+BZK (0.08% w/v)+DDM+Phosphate Buffer, (pH 7.5, Filtered, 20 mg/mL API) composition showed 87.28% Liraglutide assay and corresponding increase in peptide related impurities after 5 months storage at 2-8° C. (long-term stability) indicating instability. Total of 23 impurities were observed at ≥0.05 relative peak area (%) integrated by 215 nm UV detection.

A Milli-Q® Water+PG (11.5% v/v or 12.0% w/v)+mPEG-350+Phosphate Buffer, (pH 7.5, Filtered, 10 mg/mL API) composition showed 85.47% Liraglutide assay and corresponding increase in peptide related impurities after 8 months storage at 2-8° C. (long-term stability) indicating instability. Total of 12 impurities were observed at ≥0.05 relative peak area (%) integrated by 215 nm UV detection.

Detailed impurities data for each of the above compositions follows in Table 4.1-Table 4.5, respectively, below.

TABLE 4.1
A Milli-Q ® Water + Glycerin (11.66% or
14.7% w/v) + trehalose (0.25% w/v) + BZK
(0.08% w/v) + Phosphate Buffer, (pH 7.5, Filtered,
20 mg/mL API) - After 1 Month at 2-8° C.

| Relative | Peak Area | |
|---|---|---|
| Retention | (PA) | Peak Area |
| Time (RRT) | 215 nm | (PA) % |
| 0.938 | 9597 | 0.07 |
| 1 | 13187176 | 98.1 |
| 1.208 | 62057 | 0.46 |
| 1.387 | 8799 | 0.07 |
| 1.423 | 24742 | 0.18 |
| 1.484 | 35974 | 0.27 |
| 1.512 | 11763 | 0.09 |
| 1.522 | 20952 | 0.16 |
| 1.555 | 31717 | 0.24 |
| 1.711 | 49795 | 0.37 |
| N = 9 | 13442572 | 100 |

TABLE 4.2
A Milli-Q ® Water + PG (7.5% v/v or
7.8% w/v) + Glycerin (7.5% v/v or 9.45% w/v) +
PEG 3350 (1.0% w/v) + trehalose (2.5% w/v) +
Phosphate Buffer, (pH 7.5, Filtered,
20 mg/mL API) - After 1 Month at 2-8° C.

| Relative | Peak Area | |
|---|---|---|
| Retention | (PA) | Peak Area |
| Time (RRT) | 215 nm | (PA) % |
| 1 | 9170526 | 99.28 |
| 1.324 | 12795 | 0.14 |
| 1.391 | 11770 | 0.13 |
| 1.482 | 23485 | 0.25 |
| 1.492 | 15652 | 0.17 |
| 1.593 | 3246 | 0.04 |
| N = 5 | 9237474 | 100 |

TABLE 4.3
Milli-Q ® Water + PG (7.5% v/v or
7.8% w/v) + Glycerin (7.5% v/v or 9.45% w/v) +
PEG 3350 (1.0% w/v) + trehalose (2.5% w/v) +
Phosphate Buffer, (pH 7.5, Filtered,
20 mg/mL API) - After 7 Months at 2-8° C.

| Relative | Peak Area | |
|---|---|---|
| Retention | (PA) | Peak Area |
| Time (RRT) | 215 nm | (PA) % |
| 0.682 | 21750 | 0.26 |
| 0.964 | 12390 | 0.15 |
| 1 | 8182945 | 96.16 |
| 1.208 | 12220 | 0.14 |
| 1.31 | 44326 | 0.52 |
| 1.34 | 10895 | 0.13 |
| 1.382 | 6071 | 0.07 |
| 1.433 | 10001 | 0.12 |
| 1.472 | 64648 | 0.76 |
| 1.494 | 46211 | 0.54 |
| 1.527 | 5903 | 0.07 |
| 1.586 | 3970 | 0.05 |
| 1.598 | 28729 | 0.34 |
| 1.607 | 21182 | 0.25 |
| 1.614 | 18486 | 0.22 |
| 1.636 | 26108 | 0.31 |
| N = 15 | 8515835 | 100 |

TABLE 4.4
Milli-Q ® Water + Glycerin (11.5%
v/v or 14.5% w/v) + Dextran (6% w/v) +
trehalose (2.5% w/v) + BZK (0.08% w/v) +
DDM + Phosphate Buffer, (pH 7.5, Filtered,
20 mg/mL API) - After 5 Months at 2-8° C.

| Relative | | |
|---|---|---|
| Retention | Peak Area | Peak Area |
| Time (RRT) | (PA) 215 nm | (PA) % |
| 0.67 | 25987 | 0.36 |
| 0.953 | 14014 | 0.19 |
| 1 | 6358568 | 87.28 |
| 1.179 | 40544 | 0.56 |
| 1.248 | 9445 | 0.13 |
| 1.268 | 253118 | 3.47 |
| 1.351 | 9890 | 0.14 |
| 1.362 | 7130 | 0.1 |
| 1.38 | 92211 | 1.27 |
| 1.401 | 28974 | 0.4 |
| 1.443 | 62215 | 0.85 |
| 1.468 | 64123 | 0.88 |
| 1.485 | 7669 | 0.11 |
| 1.501 | 4862 | 0.07 |
| 1.506 | 4382 | 0.06 |
| 1.534 | 6455 | 0.09 |
| 1.541 | 11913 | 0.16 |
| 1.565 | 5865 | 0.08 |
| 1.57 | 29542 | 0.41 |
| 1.578 | 29158 | 0.4 |
| 1.583 | 88642 | 1.22 |
| 1.606 | 95477 | 1.31 |
| 1.624 | 21143 | 0.29 |
| 1.661 | 14262 | 0.2 |
| N = 23 | 7285589 | 100 |

TABLE 4.5
Milli-Q ® Water + PG (11.5% v/v
or 12.0% w/v) + mPEG-350 + Phosphate
Buffer, (pH 7.5, Filtered, 10 mg/mL API) - After 8 Months at 2-8° C.

| Relative | | |
|---|---|---|
| Retention | Peak Area | Peak Aera |
| Time (RRT) | (PA) 215 nm | (PA) % |
| 0.746 | 32085 | 0.11 |
| 1 | 23846530 | 85.47 |
| 1.326 | 24093 | 0.09 |
| 1.387 | 1590419 | 5.7 |
| 1.472 | 57021 | 0.2 |
| 1.536 | 339004 | 1.22 |
| 1.577 | 306616 | 1.1 |
| 1.617 | 501300 | 1.8 |
| 1.647 | 945561 | 3.39 |
| 1.69 | 46418 | 0.17 |
| 1.757 | 45453 | 0.16 |
| 1.785 | 137569 | 0.49 |
| 1.812 | 28483 | 0.1 |
| N = 12 | 27900552 | 100 |

Based on these results, composition of Milli-Q® Water+7.5% v/v or 7.8% w/v PG+7.5% v/v or 9.45% w/v Glycerin+1.0% w/v PEG 3350+2.5% w/v trehalose (pH 7.5) with Phosphate Buffer was selected as the Representative Composition. The pH of two different lots was found to be stable after 17 months and 12 months storage at 2-8° C. (Table 5). These two lots of this Representative Composition were tested using Reverse Phase High-Performance Liquid Chromatography combined with Ultraviolet and Mass Spectrometry (RP-HPLC-UV-MSe) approach with Waters™ UPLC CORTECS C18+ 1.6 μm column (3×150 mm), connected to BioPharmaSpec's Waters™ Acquity UPLC in-line with BioPharmaSpec's Waters™ Acquity PDA detector and Xevo G2-XS Q-TOF Mass Spectrometer for molecular weight analysis of Liraglutide composition samples in order to detect and/or identify peptide-related impurities (including UV detection) and quantify native Liraglutide content at 9 months and 15 months long-term storage (2-8° C.) that showed Liraglutide assay of 96.17% and 92.68% respectively (Table 6-7).

The respective pH of Lot 1 and Lot 2 of the Representative Composition are reflected in Table 5.

TABLE 5
pH of Lot 1 and Lot 2 of the Representative Composition.

| | Composition | pH Result | |
|---|---|---|---|
| Lot 1 | Milli-Q ® Water + 7.8% w/v PG + | T = 0: 7.63 | T = 17 Months at |
| | 9.45% w/v Glycerin + 1% w/v PEG | | 2-8° C.: 7.60 |
| | 3350 + 2.5% w/v trehalose + Phosphate | | |
| | Buffer (Filtered, pH 7.5), 20 mg/mL | | |
| Lot 2 | Milli-Q ® Water + 7.8% w/v PG+ | T = 0: 7.60 | T = 12 months at |
| | 9.45% w/v Glycerin + 1% w/v PEG | | 2-8° C.: 7.63 |
| | 3350 + 2.5% w/v trehalose + Phosphate | | |
| | Buffer (Filtered, pH 7.5), 20 mg/mL | | |

Liraglutide Assay of Lot 1 and Lot 2 of the Representative Composition are reflected in Tables 6-7 below.

TABLE 6
Lot 1.

| | | | | Timepoint/ | Native |
|---|---|---|---|---|---|
| | | | | Storage | Liraglutide |
| | Composition | Strength | pH | Temperature | Content (%) |
| Lot 1 | Milli-Q ® Water + 7.8% w/v PG + | 20 mg/mL | 7.5 | ~7M/2-8 C. | 96.16 |
| | 9.45% w/v Glycerin + 1% w/v PEG | | | ~13M/2-8 C. | 94.28 |
| | 3350 + 2.5% w/v trehalose + Phosphate | | | ~15M/2-8 C. | 92.68 |
| | Buffer (Filtered) | | | | |

TABLE 7
Lot 2.

| | | | | Timepoint/ | Native |
|---|---|---|---|---|---|
| | | | | Storage | Liraglutide |
| | Composition | Strength | pH | Temperature | Content (%) |
| Lot 2 | Milli-Q ® Water + 7.8% w/v PG + | 20 mg/mL | 7.5 | 1M/2-8 C. | 99.28 |
| | 9.45% w/v Glycerin + 1% w/v PEG | | | ~7M/2-8 C. | 97.39 |
| | 3350 + 2.5% w/v trehalose + Phosphate | | | ~9M/2-8 C. | 96.17 |
| | Buffer (Filtered) | | | | |

Detailed impurities data for the Representative Composition follows in Table 8.1-Table 8.4 below.

TABLE 8.1
Milli-Q ® Water + 7.8% w/v PG + 9.45% w/v
Glycerin + 1% w/v PEG 3350 + 2.5% w/v trehalose +
Phosphate Buffer (Filtered, pH 7.5, 20 mg/mL) -
Lot #1 After 13 Months at 2-8° C.

| Relative | | |
|---|---|---|
| Retention | Peak Area | Peak Aera |
| Time (RRT) | (PA) 215 nm | (PA) (%) |
| 0.69 | 32598 | 0.24 |
| 0.97 | 38191 | 0.28 |
| 1 | 12719547 | 94.28 |
| 1.24 | 27280 | 0.2 |
| 1.34 | 97933 | 0.73 |
| 1.38 | 42290 | 0.31 |
| 1.43 | 12961 | 0.1 |
| 1.48 | 30372 | 0.23 |
| 1.52 | 37904 | 0.28 |
| 1.53 | 14525 | 0.11 |
| 1.56 | 113796 | 0.84 |
| 1.6 | 15663 | 0.12 |
| 1.66 | 26955 | 0.2 |
| 1.67 | 18647 | 0.14 |
| 1.68 | 62346 | 0.46 |
| 1.68 | 65287 | 0.48 |
| 1.69 | 51227 | 0.38 |
| 1.71 | 75719 | 0.56 |
| 1.74 | 8114 | 0.06 |
| Sum | 13491356 | 100 |

TABLE 8.2
Milli-Q ® Water + 7.8% w/v PG + 9.45% w/v
Glycerin + 1% w/v PEG 3350 + 2.5% w/v trehalose +
Phosphate Buffer (Filtered, pH 7.5, 20 mg/mL) -
Lot #1 After 15 Months at 2-8° C.

| Relative | | |
|---|---|---|
| Retention | Peak Area | Peak Aera |
| Time (RRT) | (PA) 215 nm | (PA) (%) |
| 0.68 | 26549 | 0.27 |
| 0.79 | 5457 | 0.06 |
| 0.96 | 28767 | 0.29 |
| 1.00 | 9143606 | 92.68 |
| 1.21 | 22770 | 0.23 |
| 1.25 | 7787 | 0.08 |
| 1.31 | 72600 | 0.74 |
| 1.34 | 36990 | 0.37 |
| 1.38 | 8614 | 0.09 |
| 1.42 | 13113 | 0.13 |
| 1.43 | 27339 | 0.28 |
| 1.48 | 119979 | 1.22 |
| 1.50 | 99021 | 1.00 |
| 1.54 | 14765 | 0.15 |
| 1.58 | 6152 | 0.06 |
| 1.60 | 22535 | 0.23 |
| 1.60 | 17489 | 0.18 |
| 1.61 | 50567 | 0.51 |
| 1.61 | 43963 | 0.45 |
| 1.62 | 38155 | 0.39 |
| 1.64 | 53562 | 0.54 |
| 1.67 | 5555 | 0.06 |
| Sum | 9865335 | 100.00 |

TABLE 8.3
Milli-Q ® Water + 7.8% w/v PG + 9.45% w/v
Glycerin + 1% w/v PEG 3350 + 2.5% w/v trehalose +
Phosphate Buffer (Filtered, pH 7.5, 20 mg/mL) -
Lot #2 After 7 Months at 2-8° C.

| Relative | | |
|---|---|---|
| Retention | Peak Area | Peak Area |
| Time (RRT) | (PA) 215 nm | (PA) (%) |
| 0.68 | 30267 | 0.32 |
| 0.96 | 10088 | 0.11 |
| 1 | 9305122 | 97.39 |
| 1.21 | 8557 | 0.09 |
| 1.32 | 48708 | 0.51 |
| 1.35 | 15543 | 0.16 |
| 1.39 | 7121 | 0.07 |
| 1.45 | 10037 | 0.11 |
| 1.5 | 23451 | 0.25 |
| 1.52 | 55335 | 0.58 |
| 1.63 | 7609 | 0.08 |
| 1.64 | 13680 | 0.14 |
| 1.66 | 7277 | 0.08 |
| 1.68 | 11298 | 0.12 |
| Sum | 9554093 | 100 |

TABLE 8.4
Milli-Q ® Water + 7.8% w/v PG + 9.45% w/v
Glycerin + 1% w/v PEG 3350 + 2.5% w/v trehalose +
Phosphate Buffer (Filtered, pH 7.5, 20 mg/mL) -
Lot #1 After 9 Months at 2-8° C.

| Relative | | |
|---|---|---|
| Retention | Peak Area | Peak Area |
| Time (RRT) | (PA) 215 nm | (PA) (%) |
| 0.69 | 21422 | 0.18 |
| 0.97 | 16258 | 0.14 |
| 1.00 | 11228185 | 96.17 |
| 1.23 | 9684 | 0.18 |
| 1.29 | 7160 | 0.06 |
| 1.33 | 64893 | 0.56 |
| 1.36 | 27228 | 0.23 |
| 1.41 | 6994 | 0.06 |
| 1.46 | 12078 | 0.10 |
| 1.51 | 89955 | 0.77 |
| 1.53 | 83033 | 0.71 |
| 1.62 | 19306 | 0.17 |
| 1.63 | 11598 | 0.10 |
| 1.64 | 37908 | 0.32 |
| 1.65 | 15265 | 0.13 |
| 1.67 | 24735 | 0.21 |
| Sum | 11675702 | 100.10 |

The shelf-life prediction of these lots was performed using regression analysis by predicting the response values based on the fitted model. This statistical analysis showed that the two different lots of this representative composition (Milli-Q® Water+7.8% w/v PG+9.45% w/v Glycerin+1% w/v PEG 3350+2.5% w/v trehalose+Phosphate Buffer, pH 7.5) would be stable between ~18-21 months at long-term storage of 2-8° C. for assay to remain between 90.0-110.0%.

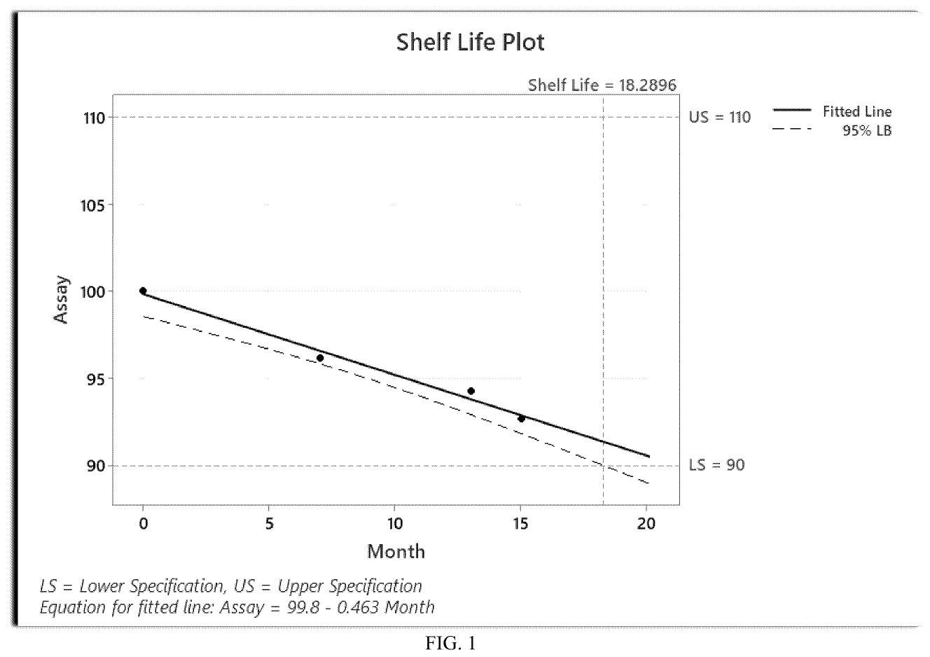

Representative Composition Lot 1: Regression Equation for Assay=99.8-0.463*Month giving predicted shelf-life of ~18 months, as illustrated by FIG. 1 in a Shelf Life Plot of the Representative Composition Lot 1.

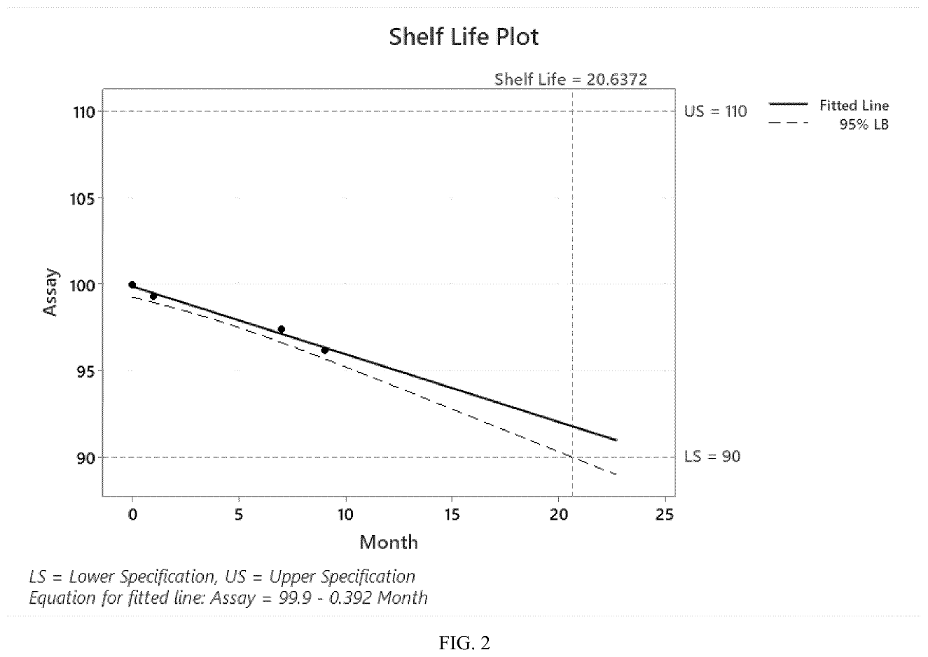

Representative Composition Lot 2: Regression Equation for Assay=99.9-0.392*Month giving predicted shelf-life of ~21 months, as illustrated by FIG. 2 in a Shelf Life Plot of the Representative Composition Lot 2.

A structural and aggregation analysis was performed of the developed composition. The two lots of the Representative Composition (Milli-Q® Water+7.8% w/v PG+9.45% w/v Glycerin+1% w/v PEG 3350+2.5% w/v trehalose+Phosphate Buffer, pH 7.5) were also evaluated after long-term storage at 2-8° C. for peptide aggregation using Size Exclusion Chromatography with UV, Refractive Index and Multi-Angle Laser Light Scattering detection (SEC-MALS) and Sedimentation Velocity Analytical Ultra Centrifugation (SV-AUC) including Ultraviolet (UV) and interference optics. Additionally, the composition was also tested for peptide structural analysis using Circular Dichroism (CD). Lot 1 of this composition was evaluated after ~15 months storage at 2-8° C.) and Lot 2 of this composition was evaluated after ~9 months storage at 2-8° C.

SEC-MALS analysis using Waters™ MaxPeak Premier SEC 250 Å 1.7 μm, 4.6×150 mm column at 0.2 mL/min on H-Class Bio Plus (with UV detector) and Wyatt DAWN™ MALS detector and Optilab™ RI detector was performed on both Lots 1 and 2 of the Representative Composition (Milli-Q® Water+7.8% w/v PG+9.45% w/v Glycerin+1% w/v PEG 3350+2.5% w/v trehalose+Phosphate Buffer, pH 7.5) showed a major signal at approximately 27 kDa, which agrees with the expected hexameric state for Liraglutide. Low detection of high molecular weight components are observed but these are below the limit of detection by UV. No significant aggregation was observed. Additionally, marketed comparator drug, Saxenda (6 mg/mL) was tested which also showed major signal at approximately 27 kDa indicating hexameric state of Liraglutide. This confirmed the chemical structural similarity of the developed compositions with Saxenda.

SV-AUC analysis using Optima Analytical Ultracentrifuge (Beckman Coulter) with an AN60Ti 4-hole rotor, Beckmann cells with sapphire windows as measurement cells and ZentriForce Pharma optical cell alignment tool was performed on both Lots 1 and 2 of the Representative Composition (Milli-Q® Water+7.8% w/v PG+9.45% w/v Glycerin+1% w/v PEG 3350+2.5% w/v trehalose+Phosphate Buffer, pH 7.5) showed an excess of Species 2 of 99.7%-99.8% with a sedimentation coefficient (s20w) of 1.41 S-1.42 S and a molecular weight of 22 kDa, which agrees with the expected hexameric state for Liraglutide. No significant aggregation was observed.

Structural analysis using CD: Analysis of secondary and tertiary peptide structure of both Lots 1 and 2 of the Representative Composition (Milli-Q® Water+7.8% w/v PG+9.45% w/v Glycerin+1% w/v PEG 3350+2.5% w/v trehalose+Phosphate Buffer, pH 7.5) using Jasco J-1500 Circular dichroism Spectropolarimeter (Near-UV and Far-UV) showed stable peptide structure of Liraglutide after formulating it in solution form.

Near-UV for Tertiary Structure Analysis: Both Lots of the Representative Composition (Milli-Q® Water+7.8% w/v PG+9.45% w/v Glycerin+1% w/v PEG 3350+2.5% w/v trehalose+Phosphate Buffer, pH 7.5) showed positive maximum peak at 272 nm in near-UV spectra. This is indicative of the oligomerization or self-assembly in ordered fashion, consistent with Liraglutide's known micelle-like aggregates. This confirms non-random, oligomeric structure of Liraglutide rather than unfolded monomer which corroborates the results of SEC-MALS and SV-AUC that showed hexameric state of Liraglutide in both Lots 1 and 2.

Far-UV for Secondary Structure Analysis: A negative band near ~208 nm together with a positive band near ~190 nm in far-UV CD spectrum indicated that the Liraglutide peptide is predominantly α-helical, which is the expected folded state for Liraglutide in solution, and has retained a native-like Glucagon-Like Peptide-1 (GLP-1)-type conformation in the Representative Composition solution (Milli-Q® Water+7.8% w/v PG+9.45% w/v Glycerin+1% w/v PEG 3350+2.5% w/v trehalose+Phosphate Buffer, pH 7.5). In summary, the far-UV results indicated that Liraglutide is in a folded, α-helical conformation, consistent with its functional oligomeric form rather than an unfolded monomer. This result corroborated with the results of SEC-MALS and SV-AUC that showed hexameric state of Liraglutide in both lots. Additionally, comparator drug product, Saxenda (6 mg/mL) was tested, which showed the same negative band near ~208 nm together with a positive band near ~190 nm in far-UV CD spectrum indicating similarity of the peptide structure of developed compositions with that of Saxenda.

Further, Circular Dichroism Second Structure (CDsstr) secondary structure contents predictions of for both lots of the Representative Composition (Milli-Q® Water+7.8% w/v PG+9.45% w/v Glycerin+1% w/v PEG 3350+2.5% w/v trehalose+Phosphate Buffer, pH 7.5) and Saxenda showed secondary structure values of 32-35% a-helix, 7-10% 3/10 helix, 5-8% β-Sheet, 10-12% turns, 7-8% poly (Pro), and 30-31% other structures. This result further confirmed the folded α-helical conformation of Liraglutide.

A mucoadhesive study was further performed using the Representative Composition (Milli-Q® Water+7.8% w/v PG+9.45% w/v Glycerin+1% w/v PEG 3350+2.5% w/v trehalose+Phosphate Buffer, pH 7.5). The study assessed and compared the drug adherence on the nasal mucosal surface (reconstructed nasal epithelial), over time. The composition was applied to the surface of the mucosal tissue and perfused/rinsed with biological buffer. The buffer was collected at specified timepoints up to 60 minutes total and the concentration of drug in the fractions was measured.

The mean amount (ng) of liraglutide retained on nasal mucosal surface is shown in Table 9, below, for Lots 1 and 2. Approximately 40-50% of Liraglutide was found to be retained on the mucosal surface after 60 minutes compared to the original applied dose for both lots of the developed composition. This indicated the mucoadhesive property of the Representative Composition (Milli-Q® Water+7.8% w/v PG+9.45% w/v Glycerin+1% w/v PEG 3350+2.5% w/v trehalose+Phosphate Buffer, pH 7.5).

TABLE 9

| | | Mean Amount | | Percent | |
|---|---|---|---|---|---|
| | | Retained (ng) | | Retained (%) | |
| | | Mean | | Mean | |
| Lot | Composition | (n = 6) | Std Err | (n = 6) | Std Err |
| Lot 1 | Milli-Q ® Water + 7.8% | 19852.40 | 928.10 | 53.55% | 2.50% |
| | w/v PG + 9.45% w/v | | | | |
| | Glycerin + 1% w/v PEG | | | | |
| | 3350 + 2.5% w/v | | | | |
| | trehalose + Phosphate | | | | |
| | Buffer (Filtered, pH 7.5, | | | | |
| | 20 mg/mL) | | | | |
| Lot 2 | Milli-Q ® Water + 7.8% | 15575.49 | 1373.81 | 40.49% | 3.57% |
| | w/v PG + 9.45% w/v | | | | |
| | Glycerin + 1% w/v PEG | | | | |
| | 3350 + 2.5% w/v | | | | |
| | trehalose + Phosphate | | | | |
| | Buffer (Filtered, pH 7.5, | | | | |
| | 20 mg/ml) | | | | |

In-Vitro Permeation Testing (IVPT) was further performed on nasal mucosal surface (reconstructed nasal epithelial) using the Representative Composition (Milli-Q® Water+7.8% w/v PG+9.45% w/v Glycerin+1% w/v PEG 3350+2.5% w/v trehalose+Phosphate Buffer, pH 7.5) and a mean flux (ng/cm²/min) vs time was measured. In the IVPT, a mean flux vs time plot shows how quickly drug permeates through skin over the course of the study. A complete IVPT curve includes lag/initial phase, rising region, peak flux and consistently declining region indicating a complete flux profile. The IVPT study of the composition (Lot 1 and Lot 2) showed lag/initial phase, rising region with no clear peak flux and consistently declining region indicating that complete flux profile was not achieved with 60-minutes of study. Consistent flux was observed throughout the 60-minute study and showed ongoing permeation of the developed composition. Table 10, below, illustrates the mean flux (ng/cm²/min) of liraglutide which permeates into receptor solution over 60 minutes following the application of the Representative Compositions.

TABLE 10

| | | Mean Peak Flux | |
|---|---|---|---|
| | | (ng/cm²/min) | |
| | | Mean | |
| Lot | Composition | (n = 4-5) | Std Error |
| Lot 1 | Milli-Q ® Water + 7.8% w/v PG + | 5.90 | 0.70 |
| | 9.45% w/v Glycerin + 1% w/v | | |
| | PEG 3350 + 2.5% w/v trehalose + | | |
| | Phosphate Buffer (Filtered, pH 7.5, | | |
| | 20 mg/mL) | | |
| Lot 2 | Milli-Q ® Water + 7.8% w/v PG + | 8.56 | 1.66 |
| | 9.45% w/v Glycerin + 1% w/v | | |
| | PEG 3350 + 2.5% w/v trehalose + | | |
| | Phosphate Buffer (Filtered, pH 7.5, | | |
| | 20 mg/mL) | | |

A cumulative amount vs. time was also measured. A desired curve starts with slow initial rise followed by the curve rising more steeply to support when flux is higher and the product is delivering the drug efficiently through the skin. Later, the curve may begin to bend/flatten as permeation slows and the system approaches depletion or steady conditions resulting in a pronounced plateau suggesting complete permeation profile. If the cumulative curve is still strongly linear/steep at the final timepoint, the study may not be long enough to characterize complete permeation profile.

The cumulative curve of the Representative Composition (Lot 1 and Lot 2) showed consistently rising profile over 60 minutes with some slow initial rise phase. The curve was still strongly linear at the end of 60 minutes timepoint indicating that complete permeation profile was not obtained. This result showed that the Representative Composition showed consistently increasing permeation. Table 11, below, illustrates the mean cumulative amount (ng) of liraglutide which permeated into receptor solution over 60 minutes following the application of the Representative Composition.

TABLE 11

| | | Mean Cumulative | |
|---|---|---|---|
| | | Amount (ng) | |
| | | Mean | |
| Lot | Composition | (n = 4-5) | Std Error |
| Lot 1 | Milli-Q ® Water + 7.8% w/v PG + | 82.50 | 6.10 |
| | 9.45% w/v Glycerin + 1% w/v | | |
| | PEG 3350 + 2.5% w/v trehalose + | | |
| | Phosphate Buffer (Filtered, pH | | |
| | 7.5, 20 mg/mL) | | |
| Lot 2 | Milli-Q ® Water + 7.8% w/v PG + | 115.63 | 23.79 |
| | 9.45% w/v Glycerin + 1% w/v | | |
| | PEG 3350 + 2.5% w/v trehalose + | | |
| | Phosphate Buffer (Filtered, pH | | |
| | 7.5, 20 mg/mL) | | |

The Representative Composition was also tested for spray pattern (using Spray View® from Proveris Scientific), droplet size distribution and dose weight (Spraytec from Malvern Panalytical) in unit-dose and multi-dose nasal spray devices.

The ovality ratio was determined from Spray Pattern testing. It is the ratio of the maximum (Dmax) to minimum (Dmin) cross-sectional diameters of the spray plume. A ratio near 1.0 indicates a nearly circular plume, which is desirable for uniform deposition in the nasal cavity upon administration. A uniform plume improves coverage of the nasal mucosa and reduces drip or runoff, contributing to consistent dosing.

Consistent and narrow droplet size distribution, which is determined based on span measurements, helps determine the suitability of the formulation composition with the nasal spray device and vice versa. Additionally, it is important to keep the percentage of droplets smaller than 10 μm to minimum as sub-10 μm droplets can pass through the nasal passages and deposit in the lower respiratory tract, which is usually unintended for products labeled for local nasal action.

As per the general pharmaceutical industry guidance, the dose weight of individual spray from nasal spray devices should be within 15% of the target dose weight and their mean dose weight within 10% of the target dose weight.

Testing with Unit-Dose Nasal Spray Devices: Ten unit-dose devices were used for spray testing to collect a data on total of 10 actuations. The mean ovality from spray pattern measurements of the final formulation composition was found to be 1.15 with a minimum of 1.09 and maximum of 1.21 indicating consistency across all ten devices. The droplet size distribution showed a span of 1.84 with a minimum of 1.75 and maximum of 1.90 indicating consistency across all ten devices. Additionally, the mean of percent sub-10 μm droplets was found to be 4.27% with a minimum of 3.35% and maximum of 4.97% indicating consistency across all ten devices. The dose weight results showed a mean of 107.0 mg with a minimum of 99.1 mg and maximum of 114.1 mg indicating consistency across all ten devices and also meeting the general requirement per industry guidance based on the target dose weight of 100 mg. These results are reflected in Table 12 and Table 13 below.

TABLE 12

| | | Average Droplet Size | | | | |
|---|---|---|---|---|---|---|
| Unit- | Dose | Distribution (DSD) Results | | | | |
| Dose Nasal | Weight | Dv10 | Dv50 | Dv90 | | % of |
| Spray Device | (mg) | (μm) | (μm) | (μm) | Span | <10 μm |
| Minimum | 99.1 | 12.53 | 27.70 | 63.47 | 1.75 | 3.35 |
| (n = 10) | | | | | | |
| Maximum | 114.1 | 14.08 | 31.20 | 73.15 | 1.90 | 4.97 |
| (n = 10) | | | | | | |
| Mean | 107.0 | 13.18 | 29.28 | 67.19 | 1.84 | 4.27 |
| (n = 10) | | | | | | |

TABLE 13

| | Spray Pattern Results | | |
|---|---|---|---|
| Unit-Dose Nasal | Dmin | Dmax | |
| Spray Device | (mm) | (mm) | Ovality |
| Minimum (n = 10) | 24.26 | 27.70 | 1.09 |
| Maximum (n = 10) | 28.63 | 33.88 | 1.21 |
| Mean (n = 10) | 27.04 | 31.12 | 1.15 |

Testing with Multi-Dose Nasal Spray Devices: Two multi-dose devices were used for spray testing to collect a data on total of 20 actuations (10 actuations per device). Two types of multi-dose devices were tested.

Multi-Dose Device #1: The mean ovality from spray pattern measurements of the final optimized formulation composition was found to be 1.32 with a minimum of 1.17 and maximum of 1.72 indicating consistency across all ten devices. The droplet size distribution showed a span of 1.70 with a minimum of 1.63 and maximum of 1.79 indicating consistency across all ten devices. Additionally, the mean of percent sub-10 μm droplets was found to be 0.37% with a minimum of 0.26% and maximum of 0.48% indicating consistency across all ten devices. The dose weight results showed a mean of 109.80 mg with a minimum of 107.20 mg and maximum of 111.0 mg indicating consistency across all ten devices and also meeting the general requirement per industry guidance based on the target dose weight of 100 mg. These results are reflected in Table 14 and Table 15 below.

TABLE 14

| Preservative- | | Average Droplet Size | | | | |
|---|---|---|---|---|---|---|
| Free Nasal | Dose | Distribution (DSD) Results | | | | |
| Spray Multi- | Weight | Dv10 | Dv50 | Dv90 | | Droplets |
| Dose Device #1 | (mg) | (μm) | (μm) | (μm) | Span | <10 μm (%) |
| Minimum | 107.2 | 30.77 | 74.95 | 157.50 | 1.63 | 0.26 |
| (n = 10) | | | | | | |
| Maximum | 111.0 | 34.90 | 84.39 | 177.40 | 1.79 | 0.48 |
| (n = 10) | | | | | | |
| Mean | 109.8 | 33.00 | 80.00 | 168.00 | 1.70 | 0.37 |
| (n = 10) | | | | | | |

TABLE 15

| Preservative-Free | Spray Pattern Results | | |
|---|---|---|---|
| Nasal Spray Multi- | Dmin | Dmax | |
| Dose Device #1 | (mm) | (mm) | Ovality |
| Minimum (n = 20) | 13.03 | 16.76 | 1.17 |
| Maximum (n = 20) | 17.54 | 24.80 | 1.72 |
| Mean (n = 20) | 15.50 | 20.28 | 1.32 |

Multi-Dose Device #2: The mean ovality from spray pattern measurements of the final optimized formulation composition was found to be 1.15 with a minimum of 1.09 and maximum of 1.22 indicating consistency across all ten devices. The droplet size distribution showed a span of 1.90 with a minimum of 1.84 and maximum of 2.02 indicating consistency across all ten devices. Additionally, the mean of percent sub-10 μm droplets was found to be 1.08% with a minimum of 0.91% and maximum of 1.26% indicating consistency across all ten devices. The dose weight results showed a mean of 107.70 mg with a minimum of 106.70 mg and maximum of 108.0 mg indicating consistency across all ten devices and also meeting the general requirement per industry guidance based on the target dose weight of 100 mg. These results are reflected in Table 16 and Table 17 below.

TABLE 16

| Preservative- | | Average Droplet Size | | | | |
|---|---|---|---|---|---|---|
| Free Nasal | Dose | Distribution (DSD) Results | | | | |
| Spray Multi- | Weight | Dv10 | Dv50 | Dv90 | | Droplets |
| Dose Device #2 | (mg) | (μm) | (μm) | (μm) | Span | <10 μm (%) |
| Minimum | 106.7 | 20.46 | 47.02 | 107.20 | 1.84 | 0.91 |
| (n = 10) | | | | | | |
| Maximum | 108.0 | 23.53 | 57.76 | 134.20 | 2.02 | 1.26 |
| (n = 10) | | | | | | |
| Mean | 107.7 | 22.00 | 53.00 | 123.00 | 1.90 | 1.08 |
| (n = 10) | | | | | | |

TABLE 17

| Preservative-Free | Spray Pattern Results | | |
|---|---|---|---|
| Nasal Spray Multi- | Dmin | Dmax | |
| Dose Device #2 | (mm) | (mm) | Ovality |
| Minimum (n = 20) | 20.64 | 24.32 | 1.09 |
| Maximum (n = 20) | 22.90 | 25.92 | 1.22 |
| Mean (n = 20) | 21.83 | 25.08 | 1.15 |

While this invention has been described with reference to examples thereof, it shall be understood that such description is by way of illustration only and should not be construed as limiting the scope of the claimed examples. Accordingly, the scope and content of the examples are to be defined only by the terms of the following claims. Furthermore, it is understood that the features of any example discussed herein may be combined with one or more features of any one or more examples otherwise discussed or contemplated herein unless otherwise stated.

What is claimed is:

1. A pharmaceutical composition for intranasal administration comprising:
an intranasal Liraglutide composition comprising Propylene Glycol (PG), high molecular weight Polyethylene Glycol (PEG), Glycerin, trehalose and water, and having a pH of 7.0 to 8.4.

2. The composition of claim 1, wherein the pH of the intranasal Liraglutide composition is 7.0 to 8.0.

3. The composition of claim 1, wherein the pH of the intranasal Liraglutide composition is 7.2 to 8.0.

4. The composition of claim 1, wherein the pH of the intranasal Liraglutide composition is 7.4 to 7.6.

5. The composition of claim 1, wherein the pH of the intranasal Liraglutide composition is 7.3 to 7.7.

6. The composition of claim 5, wherein the intranasal Liraglutide composition further comprises a buffer.

7. The composition of claim 6, wherein the buffer is a phosphate buffer.

8. The composition of claim 7, wherein the high molecular weight Polyethylene Glycol (PEG) is Polyethylene Glycol 3350 (PEG 3350).

9. The composition of claim 8 comprising between 5% to 15% v/v or 5.2% to 15.6% w/v Propylene Glycol (PG), 5% to 15% v/v or 6.3% to 18.9% w/v Glycerin, and 0.3% to 1.0% w/v Polyethylene Glycol (PEG 3350), between 0.25% and 10% w/v of trehalose and, optionally, up to 0.08% w/v of Benzalkonium chloride (BZK).

10. The composition of claim 9, wherein the intranasal Liraglutide composition exhibits less than 10% total Liraglutide impurities when stored at 2-8° C. for up to at least 15 months.

11. The composition of claim 9, wherein the intranasal Liraglutide composition exhibits less than 10% total Liraglutide impurities when stored at 2-8° C. for up to at least 9 months.

12. The composition of claim 11, wherein the total Liraglutide impurities are less than 5%.

13. The composition of claim 9, wherein the intranasal Liraglutide composition maintains an assay between 90.0-110.0% when stored at 2-8° C. for up to at least 9 months.

14. The composition of claim 13, wherein the pH of the intranasal Liraglutide composition is 7.5-7.7.

15. The composition of claim 14, wherein the pH of the intranasal Liraglutide composition is 7.5.

16. The composition of claim 14, wherein the pH of the intranasal Liraglutide composition is 7.6.

17. The composition of claim 14, wherein the intranasal Liraglutide composition maintains an assay between 90.0-110.0% when stored at 2-8° C. for up to at least 9 months.

18. The composition of claim 14 comprising 7.5% v/v or 7.8% w/v Propylene Glycol (PG), 7.5% v/v or 9.45% w/v Glycerin, and 1% w/v Polyethylene Glycol (PEG 3350) and 2.5% w/v trehalose.

19. The composition of claim 18, wherein the intranasal Liraglutide composition exhibits less than 10% total Liraglutide impurities when stored at 2-8° C. for up to at least 15 months.

20. The composition of claim 18, wherein the intranasal Liraglutide composition exhibits less than 10% total Liraglutide impurities when stored at 2-8° C. for up to at least 9 months.

21. The composition of claim 20, wherein the total Liraglutide impurities are less than 5%.

22. The composition of claim 18, wherein the intranasal Liraglutide composition maintains an assay between 90.0-110.0% when stored at 2-8° C. for up to at least 9 months.

23. An intranasal Liraglutide composition comprising:
water, between 5% and 15% v/v Propylene Glycol (PG), between 5% and 15% v/v Glycerin, between 0.3% and 1.0% w/v high molecular weight polyethylene glycol (PEG 3350), between 0.25% and 10% w/v trehalose, a pH of 7.2 to 7.8 maintained with a phosphate buffer, and optionally up to 0.08% w/v of Benzalkonium chloride (BZK).
